(19) United States
(12) Patent Application Publication
(10) Pub. No.: US 2004/0147909 A1
(43) Pub. Date: Jul. 29, 2004

(54) Surgical instrument

(75) Inventors: Constance Elaine Johnston, Eads, TN (US); Phillip Andrew Ryan, Memphis, TN (US); Perry Robin Mykleby, Collierville, TN (US)

Correspondence Address: OLIFF & BERRIDGE, PLC, P.O. BOX 19928, ALEXANDRIA, VA, 22320, US

(73) Assignee: GYRUS ENT L.L.C., BARTLETT, TN

(21) Appl. No.: 10/703,456

(22) Filed: Nov. 10, 2003

Related U.S. Application Data
(60) Provisional application No. 60/434,648, filed on Dec. 20, 2002.

(51) Int. Cl. B05B 1/00; A61B 17/00

(52) U.S. Cl. 606/1; 156/279

(57) ABSTRACT

A surgical instrument comprises a probe (3) including an elongate hollow outer member (14) having a cutting window (16) at its distal end portion. An elongate drive shaft (15) is disposed within the outer member (14) and is mounted for rotation about its longitudinal axis within the probe. A cutting tool (17) is located at the distal end of the drive shaft, and is positioned adjacent to the cutting window (16). An indicia in the form of an etched marking (18) runs proximally along the outer member (14) in alignment with the center of the cutting window (16). The indicia allows the user of the instrument to ascertain the position of the cutting window (16), whatever the orientation of the probe (3).

This nonprovisional application claims the benefit of U.S. Provisional Application No. 60/434,648, filed Dec. 20, 2002.

This invention relates to a surgical instrument such as a shaver or burr in which a surgical tool is rotatably driven by a handpiece. Such instruments typically include a rigid outer tube within which an inner tube is rotated, for example by a motor. A cutting implement, such as a cutting blade or abrading burr, is disposed on the distal end of the inner tube. Tissue or bone is exposed to the cutting implement through an opening in the distal end of the outer tube, and fragments cut by the cutting implement are drawn through the interior of the inner tube by the use of suction. Such instruments can employ tubes that are straight or curved, and U.S. Pat. No. 5,755,731 is a typical example of such a device.

Devices such as these are designed to be employed in surgery in which the instrument is inserted into a confined space within the patient's body. An example of this type of surgery is endoscopic surgery, in which the surgical site is viewed by means of an endoscope. In other surgical procedures, such as many ENT procedures, the surgeon is only able to maintain a relatively limited view of the instrument, due to the confined space in which the procedure has to be carried out. During the surgery, it is often necessary for the surgeon to rotate the device such that the cutting window is in different orientations. The present invention attempts to provide an improvement to the surgeon in maintaining a rapid appreciation of the orientation of the instrument.

Accordingly there is provided a surgical instrument comprising a handpiece, an elongate surgical tool having an axis and being rotatably situated within an outer member, the outer member having a cutting window facing at least partially in a direction transverse to the axis, the surgical instrument being provided with an indicia proximal of the cutting window and in alignment therewith such that a user of the instrument is given an indication of the rotational position of the cutting window. In this way, the orientation of the cutting window can be ascertained by the surgeon, even if the window itself is obscured.

The indicia is conveniently provided on the outer surface of the outer member, preferably towards or at the proximal end thereof. In one arrangement the indicia extends for all or a substantial part of the distance between the cutting window and the proximal end of the outer member. The indicia therefore provides an indication of the cutting window orientation which is quick and easy for the surgeon to see, even if the distal end of the instrument is obscured.

The indicia is conveniently a physical mark applied to the outer member, typically an etched mark, or alternatively as a printed mark. Conceivably the mark is a simple line or arrow, preferably in alignment with the center of the cutting window. Other more extravagant markings, such as company names or logos, can also be employed as indicia to indicate the cutting window orientation.

The use of the indicia in accordance with the present invention is particularly valuable in instruments in which wherein the outer member is a straight cylindrical tube. These straight blade instruments are those in which it is most difficult to ascertain the orientation of the cutting window. In a preferred embodiment, the outer member is attached to the handpiece by means of a swivel collet, which allows the rotation of the outer member with respect to the handpiece. A swivel collet, such as that described in U.S. Pat. No. 5,492,527 and in our co-pending patent application U.S. Ser. No. 10/103,104, allows the outer member and hence the cutting window to be easily rotated with respect to the instrument handpiece. In this instance, it is particularly important to keep track of the orientation of the cutting window, and the present invention has particular application with this type of instrument.

The invention will now be described in greater detail, by way of example, with reference to the drawings, in which:-

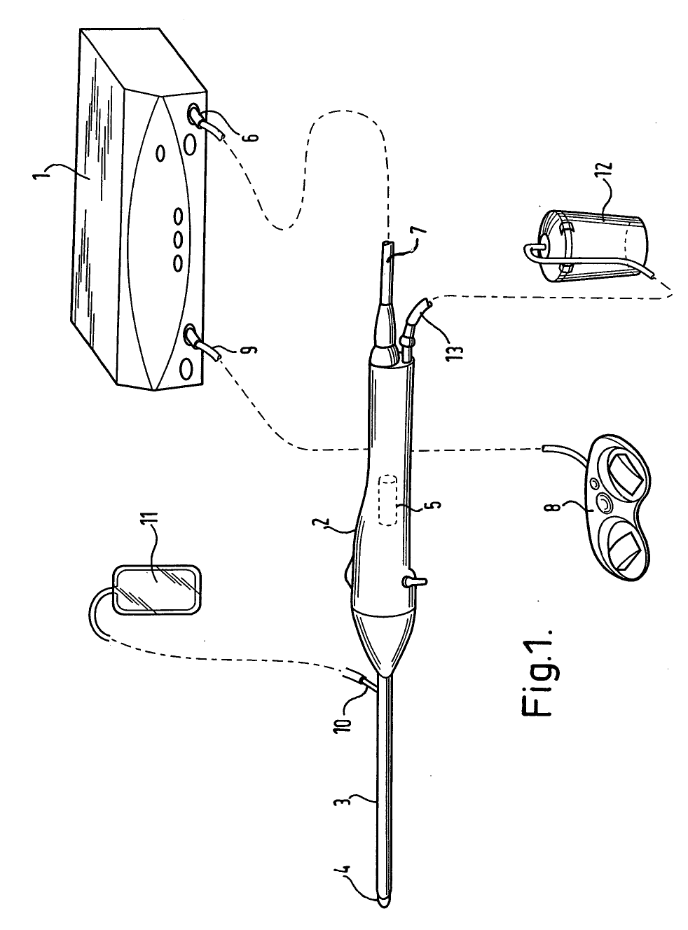

FIG. 1 is a schematic diagram of a surgical system incorporating a surgical instrument in accordance with the invention;

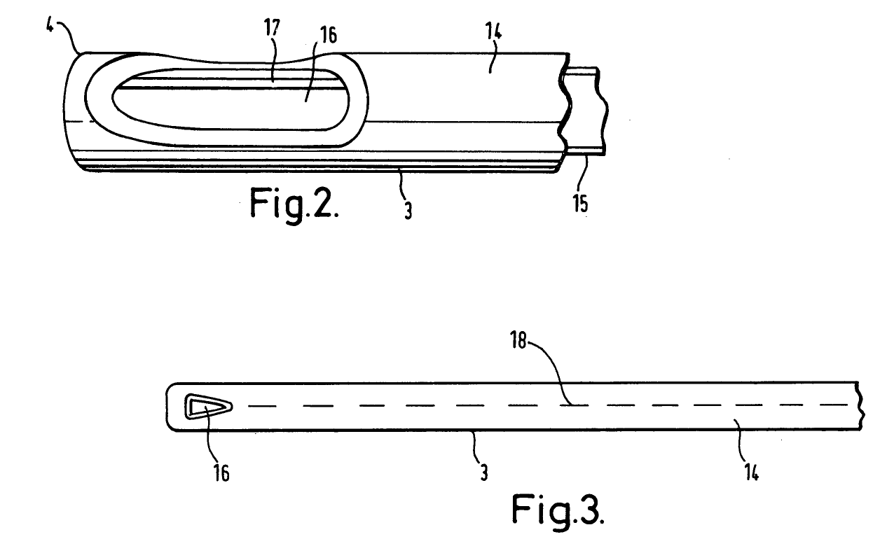

FIG. 2 is a side view, partly in section, of the distal end of the surgical instrument of FIG. 1;

FIG. 3 is a side view of the surgical probe of the instrument of FIG. 1;

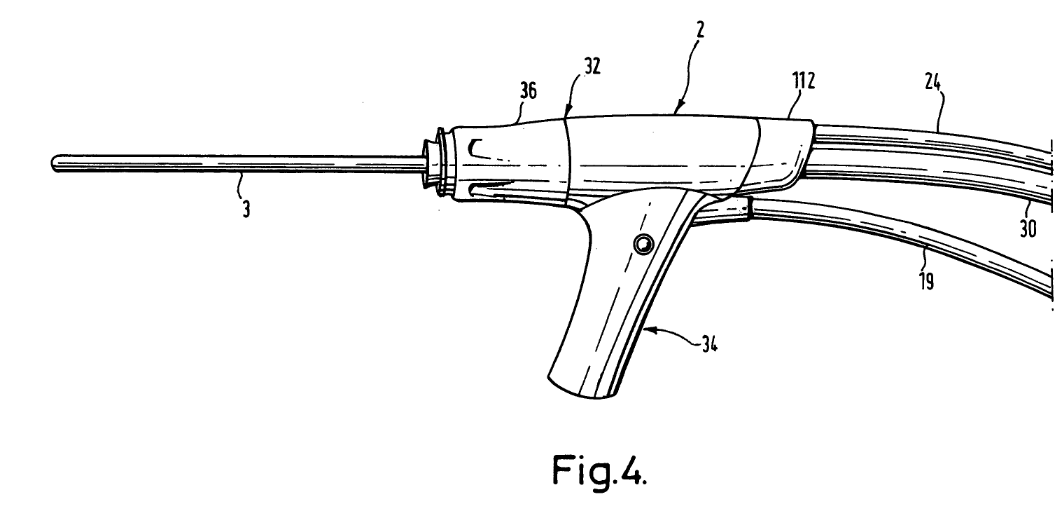

FIG. 4 is a side view of a surgical instrument in accordance with an alternative embodiment of the invention;

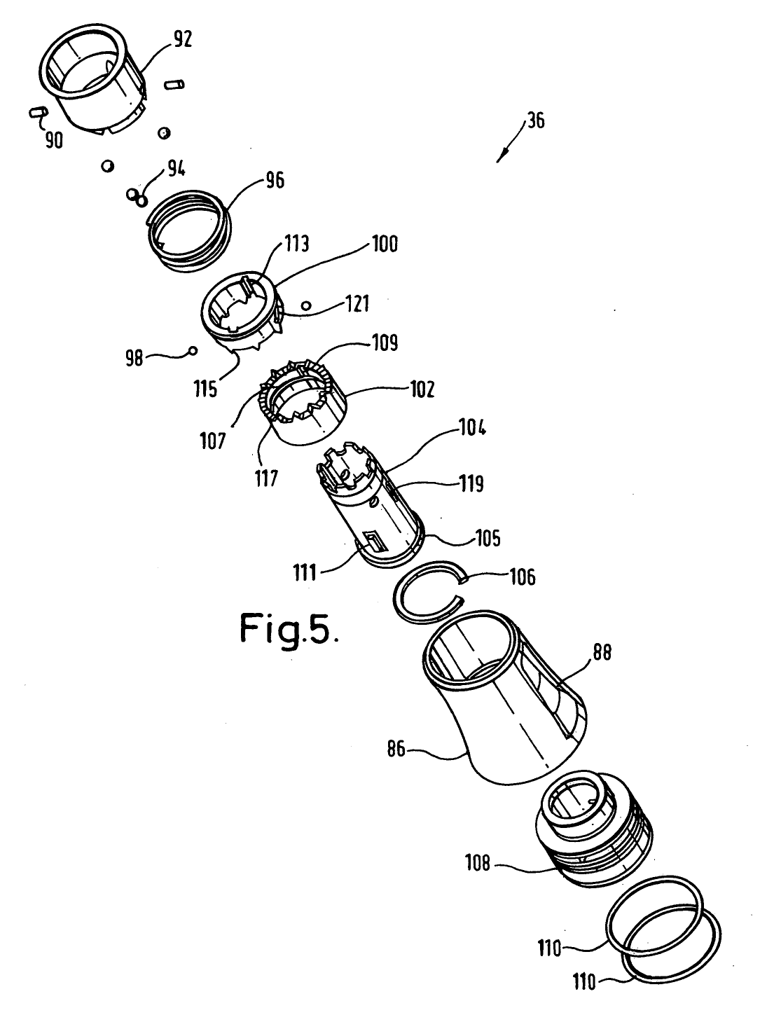

FIG. 5 is an exploded diagram showing the parts making up the collet assembly of the device of FIG. 4;

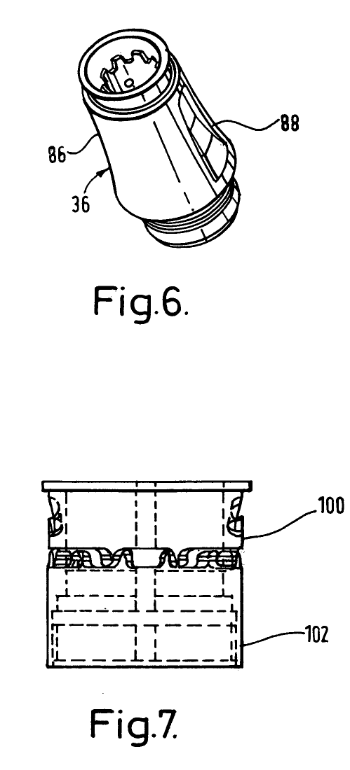

FIG. 6 is a perspective view of the swivel collet of FIG. 5; and

FIG. 7 is a side view, with hidden detail, of various parts of the swivel collet of FIG. 5.

Referring to the drawings, FIG. 1 shows a surgical system which includes a controller 1 and a handpiece 2 having a detachable surgical probe shown generally at 3. The probe 3 comprises an outer tubular member 14 and an inner tubular member 15. The inner member 15 is driven by a motor shown schematically at 5 within the handpiece. Power signals for the motor 5 are supplied to the handpiece 2 from an output socket 6 on the generator 1, via connector cord 7. Activation of the controller 1 may be performed by means of footswitch 8, coupled to the controller by means of connector cord 9. An inlet port 10 allows saline to be fed from a saline source 11 to the distal end of the probe 3. A source of suction 12 is also provided, coupled to the handpiece by cord 13.

FIG. 2 shows a close up of the distal end 4 of the probe 3. The outer member 14 is provided with a lateral cutting window 16 through which a cutting tool 17 located at the distal end of the inner member 15 is accessed. In use, the probe 3 is moved to engage tissue to be excised, and the tissue is drawn into the cutting window by the suction applied through the inner tubular member 15. When the tissue enters the cutting window 16, it is severed by the rotation of the cutting tool 17 and the excised tissue is evacuated by the suction along the inner member 15.

FIG. 3 shows a probe 3 and the cutting window 16. Running proximally along the outer member 14 in alignment with the center of the cutting window is an indicia in the form of an etched marking 18. The etched marking 18 extends to the proximal end of the outer member 14, to where the outer member is attached to the handpiece 2. Thus the user of the instrument is able to ascertain the positioning of the cutting window 16, even if it is not readily visible to the user.

FIG. 4 shows an alternative embodiment of surgical device in which the handpiece 2 includes an upper portion 32 and a lower portion 34 defining a pistol grip arrangement. The upper portion 32 extends generally parallel to the probe 3, while the lower portion 34 extends at an angle thereto. The probe 3 is attached to the upper portion of the handpiece 2 by means of a collet assembly 36. The motor 5 (not shown in FIG. 4) is located in the lower portion 34 of the handpiece, and is controlled by signals via control line 19. Fluid irrigation and suction are provided to the handpiece 2 via dual tubing 31, the fluid supply being via tube 24 and the suction supply via tube 30. The dual tubing 31 is attached to the handpiece 2 by means of a connector 112.

The swivel collet assembly 36 is shown in more detail in FIGS. 5 to 7. As shown in FIG. 4, the collet assembly 36 is provided at the front end of the upper portion 32 of the handle 2. Disposing the collet assembly 36 at this location enables an operator, such as a surgeon, holding the handle 2 in a pistol grip manner, to touch and rotate the assembly collet 36 or a portion thereof with the tip of at least one of the surgeon's fingers. Rotating at least a portion of the collet assembly 36 in this manner enables the cutting window of the probe 3 to rotate, thereby orienting the direction of the shaving and/or cutting of the desired bodily material.

As shown in FIGS. 5 and 6, the collet assembly 36 includes a swivel shell 86 that defines at least one gripping channel 88. The at least one gripping channel 88 enhances the surgeon's ability to grip the collet assembly 36 with the tip of at least one of the surgeon's fingers so as to rotate at least a part of the collet assembly 36. FIGS. 5 and 7 show a combination of sub-elements that enable manual rotation of the swivel shell 86 to change the orientation of the cutting window while the inner blade of the probe 3 rotates. The collet assembly includes release pins 90, a release ring 92, retention balls 94, a lock spring 96, unlocking balls 98, a sliding cam 100, a stationary cam 102, a retention sleeve 104, a retaining clip 106, the swivel shell 86, a base mount 108, and base mount seals 110.

The collet assembly includes a stationary cam 102 which is attached to the base mount 108 such that an interior gap defines a location for the retention of a flange 105 on the proximal end of the retention sleeve 104, thus capturing the retention sleeve and preventing it from moving axially, but allowing it to rotate freely and concentrically with respect to the main axis of the collet assembly. One method of capturing the flange on the retention sleeve is to use a retaining clip 106 which fits into an internal groove 107 in the stationary cam and defines a gap which ensures that rotation is free, but that axial movement is restricted. The use of the retaining clip further facilitates the assembly of the mechanism, by allowing the base mount 108 to be assembled into contact with the retaining clip 106 thereby setting the relative position of the base mount to the stationary cam and eliminating the need to adjust this engagement by manual means.

Two interior grooves 109 are located on the stationary cam 102 to provide relief to allow the cam to slide over two keys 111 on the exterior of the retention sleeve. These two grooves are provided as a means to aid assembly and are not functional once the collet assembly 36 has been completed. The sliding cam 100 also has two interior grooves 113 which engage with the keys 111 on the exterior of the retention sleeve 104 preventing relative rotational motion of these parts, but allowing the sliding cam 100 to slide freely in an axial direction along the length of the retention sleeve 104. This engagement is the means by which rotational motion is transmitted between the sliding cam 100 and the retention sleeve 104 and subsequently to the blade hub when the swivel shell 86 is rotated. The sliding cam engages 100 with the stationary cam 102 by means of teeth 115 and 117 that are located on the faces of each part facing towards each other. The teeth 115 and 117 are held in engagement by the spring 96 which is in turn retained by the release ring 92 which is retained by the release pins 90 which are engaged in holes in the release ring 92 and whose ends are placed in slots 119 in the retention sleeve 104. The release pins 90 are retained by the assembly of the swivel shell 86 which prevents the pins from falling out the holes which capture them in the release ring 92.

The teeth 115 and 117 on the cams 100 and 102 that engage with each other have geometry which when urged into engagement by the lock spring 96, are not permitted to slide against each other by means of friction. In order to prevent sliding of the teeth against other the contact angle of the teeth is substantially less than 45 degrees and in this case is 15 degrees. The grooves 121 on the exterior of the sliding cam 100 are shaped with a V profile and receive the unlocking balls 98 which engage in pockets inside the swivel shell 86. The balls 98 slide in the V shaped grooves 121 in the sliding cam 100 when the swivel shell 86 is rotated. Rotation of the swivel shell 86 by the surgeon causes a corresponding rotation of the sliding cam 100, lifting the sliding cam 100 out of engagement with the stationary cam 102. Once the sliding cam is free from the stationary cam, it can cause a corresponding rotation of the retention sleeve 104. In this way, a rotation of the swivel shell 86 causes a reorientation of the cutting window in the probe 3, via retention sleeve 104. However, should the retention sleeve be urged to rotate, for example by the probe 3, the rotation will be prevented by the engagement of the sliding cam 100 in the stationary cam 102. The action of the swivel shell 86 to lift the sliding cam 100 out of engagement with the stationary cam 102 means that while a rotation of the swivel shell will cause a corresponding rotation of the retention sleeve 104, the reverse will not be permitted (i.e. an attempt to rotate the retention sleeve 104 will not cause a corresponding rotation of the swivel shell 86). This provides the assurance that in the event of a jam the swivel shell will be prevented from rotating, thereby avoiding the possibility of injury to the surgeon.

As will be seen from the above, the surgeon can easily rotate the probe 3 by rotating the swivel shell 86. Thus the orientation of the cutting window 16 with respect to the handpiece 2 can vary during a surgical procedure. The provision of the etched marking 18 on the outer member 14 of the probe 3 enables the surgeon to keep track of the orientation of the probe 3, and hence the cutting window 16.

1. A surgical instrument comprising a handpiece, an elongate surgical tool having an axis and being rotatably situated within an outer member, the outer member having a cutting window facing at least partially in a direction transverse to the axis, the surgical instrument being provided with an indicia proximal of the cutting window and in alignment therewith such that a user of the instrument is given an indication of the rotational position of the cutting window.

2. A surgical instrument according to claim 1 wherein the indicia is provided on the outer surface of the outer member.

3. A surgical instrument according to claim 1 wherein the indicia is provided towards the proximal end of the outer member.

4. A surgical instrument according to claim 1 wherein the indicia is provided at the proximal end of the outer member.

5. A surgical instrument according to claim 1 wherein the indicia extends for a substantial part of the distance between the cutting window and the proximal end of the outer member.

6. A surgical instrument according to claim 5 wherein the indicia extends continuously from the proximal end of the outer member to the cutting window.

7. A surgical instrument according to claim 1 wherein the indicia is a physical mark applied to the outer member.

8. A surgical instrument according to claim 1 wherein the indicia is an etched mark.

9. A surgical instrument according to claim 1 wherein the indicia is a printed mark.

10. A surgical instrument according to claim 1 wherein the indicia is a mark in alignment with the center of the cutting window.

11. A surgical instrument according to claim 1 wherein the outer member is a straight cylindrical tube.

12. A surgical instrument according to claim 1 wherein the outer member is attached to the handpiece by means of a swivel collet, which allows the rotation of the outer member with respect to the handpiece.
